(19) United States
(12) Patent Application Publication
(10) Pub. No.: US 2025/0334122 A1
(43) Pub. Date: Oct. 30, 2025

(54) PUMP UNIT FOR A CENTRIFUGAL PUMP AND A CENTRIFUGAL PUMP

(71) Applicant: Levitronix GmbH, Zürich (CH)

(72) Inventors: Daniel Steinert, Bülach (CH); Rennan Hu, Zürich (CH); Alexander Schmid, Hausen am Albis (CH); Natale Barletta, Zürich (CH)

(21) Appl. No.: 19/095,563

(22) Filed: Mar. 31, 2025

(30) Foreign Application Priority Data
Apr. 30, 2024 (EP) 24173505.9

(51) Int. Cl. F04D 13/06; F04D 1/00; F04D 29/42

(52) U.S. Cl. CPC F04D 13/06; F04D 1/00; F04D 29/4293

(57) ABSTRACT

A pump unit includes a pump housing with an inlet and an outlet and a rotor arranged in the pump housing. The pump unit is designed for a non-contact magnetic levitation of the rotor and for a non-contact magnetic drive of the rotor by the stator. The vanes of the rotor are arranged around a central inlet area of the rotor, and the rotor includes a relief opening for generating a recirculation flow which is directed from a back side of the rotor facing away from the inlet in the direction of the central inlet area of the rotor, and a separating element arranged in the central inlet area, which redirects the recirculation flow in a radial direction perpendicular to the axial direction. The, the separating element and the relief opening of the rotor are a one-piece unit.

### CROSS-REFERENCE TO RELATED APPLICATION

This application claims priority to European Application No. 24173505.9, filed Apr. 30, 2024, the contents of which are hereby incorporated herein by reference.

### BACKGROUND

### Technical Field

The disclosure relates to a pump unit for a centrifugal pump. The disclosure further relates to a centrifugal pump with such a pump unit.

### Background Information

Centrifugal pumps are known which comprise a pump unit and a stator which is designed as a drive unit for the rotor of the pump unit, wherein the rotor of the pump unit forms the centrifugal wheel of the centrifugal pump. The rotor can be magnetically supported without contact and can be driven without contact to rotate about an axial direction by the stator in the pump unit. Such centrifugal pumps are marketed, for example, by the applicant under the product name Levitronix® BPS pumps.

The stator and the rotor generally form an electromagnetic rotary drive. In the Levitronix® BPS pumps, for example, the electromagnetic rotary drive is designed according to the principle of the bearingless motor. The term bearingless motor refers to an electromagnetic rotary drive in which the rotor can be supported completely magnetically with respect to the stator, wherein no separate magnetic bearings are provided. For this purpose, the stator is designed as a bearing and drive stator, which is both the stator of the electric drive and the stator of the magnetic bearing. A magnetic rotating field can be generated with the electrical windings of the stator, which on the one hand exerts a torque on the rotor, which effects its rotation about a desired axis of rotation defined by the axial direction and which, on the other hand, exerts an arbitrarily adjustable transverse force on the rotor so that its radial position can be actively controlled or regulated. Thus, three degrees of freedom of the rotor can be actively regulated, namely its rotation and its radial position (two degrees of freedom).

### SUMMARY

With respect to three further degrees of freedom, namely its position in axial direction and tilting with respect to the radial plane perpendicular to the desired axis of rotation (two degrees of freedom), the rotor is passively magnetically supported or stabilized by reluctance forces, i.e., it cannot be controlled. The absence of a separate magnetic bearing with a complete magnetic bearing of the rotor is the property, which gives the bearingless motor its name. In the bearing and drive stator, the bearing function cannot be separated from the drive function.

Of course, other designs of centrifugal pumps are also known in which the rotor is magnetically supported without contact, for example those in which separate magnetic bearings are provided for the rotor so that the magnetic bearing function is separated from the drive function. For example, separate coils are provided for this purpose, with which only the bearing forces for the rotor are realized, but which do not contribute to the drive of the rotor. For example, such a centrifugal pump is disclosed in WO 2022/004144.

Centrifugal pumps with non-contact magnetically supported and driven rotors, for example those which are designed and operated according to the principle of the bearingless motor, have proven themselves in a large number of applications. Due to the absence of mechanical bearings, such centrifugal pumps are suitable for applications in which very sensitive substances are conveyed, for example blood pumps, or on which very high demands are made with respect to purity, for example in the semiconductor industry, the pharmaceutical industry, the biotechnological industry, or with which abrasive or aggressive substances are conveyed, which would very quickly destroy mechanical bearings, for example pumps for slurry, sulfuric acid, phosphoric acid or other chemicals in the semiconductor industry.

FIG. 1 shows a view of a centrifugal pump 200′ known from the state of the art, which is designed according to the principle of the bearingless motor. This is a Levitronix® BPS pump, for example. For better understanding, a segment has been cut out in FIG. 1 so that the inside of the centrifugal pump 200′ is visible. The centrifugal pump 200′ comprises a stator 100′ and a pump unit 1′.

A pump unit 1′ which is suitable for this type of centrifugal pumps 200′ is disclosed, for example, in EP 2 273 124 A1. FIG. 2 shows such a pump unit 1′ in a sectional view, wherein the section is made in the axial direction A.

To indicate that the representation in FIG. 1, FIG. 2 are devices from the state of the art, the reference signs are each marked here with an inverted comma or with a dash. The centrifugal pump is designated in its entirety by the reference sign 200′.

A rotor 10′ is arranged in the pump unit 1′, which forms the centrifugal wheel or the impeller with which the fluid is conveyed. The stator 100′ has a stator housing 130′ and extends in an axial direction A from a first axial end 110′ to a second axial end 120′, wherein a cup-shaped recess 121′ is provided at the first axial end 110′, into which recess the pump unit 1′ can be inserted. The stator 100′ together with the rotor 10′ forms an electromagnetic rotary drive for rotating the rotor 10′ about the axial direction A. The stator 100′ is designed for non-contact magnetic bearing of the rotor 10′ according to the principle of the bearingless motor. For this purpose, the stator 100′ is designed as a bearing and drive stator, with which the rotor 10′ can be magnetically driven without contact for rotation about the axial direction A and can be magnetically supported without contact with respect to the stator 100′, wherein the rotor 10′ is passively magnetically stabilized with respect to the axial direction A and is actively magnetically supported in a radial plane perpendicular to the axial direction A, which plane is indicated by the line E in FIG. 1.

The electromagnetic rotary drive with the stator 100′ and the rotor 10′ is designed as a so-called temple motor. The stator 100′ comprises a plurality of coil cores 125′, here eight coil cores 125′, each of which comprises a longitudinal leg 126′, which extends from a first end, in FIG. 1 the lower end according to the representation, in the axial direction A to a second end, and a transverse leg 127′, which is arranged at the second end of the longitudinal leg 126′ and in the radial plane E. Each transverse leg 127′ extends from the associated longitudinal leg 126′ in radial direction towards the rotor 10′ and is delimited by a radially inside located end face. The coil cores 126′ are arranged around the cup-shaped recess 121′ with respect to the circumferential direction and thus around the rotor 10′, so that the rotor 10′ is arranged between the radially inside located end faces of the transverse legs 127′ of the coil cores 126′.

All first ends of the longitudinal legs 126′ are connected to one another by a back iron 122′ for conducting the magnetic flux. At least one concentrated winding 160′, 161′ is provided at each longitudinal leg 126′, which surrounds the respective longitudinal leg 126′. With respect to the number and arrangement of the concentrated windings 160′, 161′, many variants are known, which are not explained in more detail here. For example, there are such windings 160′ which are wound around exactly one longitudinal leg 126′ and such windings 161′ which are arranged around exactly two longitudinal legs 126′.

The plurality of the longitudinal legs 126′, which extend in the axial direction A and are reminiscent of the columns of a temple has given the temple motor its name.

The pump unit 1′ which is known from EP 2 273 124 A1 (FIG. 2) comprises a pump housing 2′ with an inlet 21′ and with an outlet 22′ for the fluid to be conveyed, as well as the rotor 10′ arranged in the pump housing 2′ for conveying the fluid, which rotor can be rotated about the axial direction A. The rotor 10′ comprises a magnetically effective core 101′, which cooperates magnetically with the stator 100′ to generate the torque as well as to generate the magnetic bearing forces. For example, the magnetically effective core 101′ is a permanent magnetic ring or a permanent magnetic disk.

Such designs are also possible in which the magnetically effective core 101′ is designed in a permanent magnetic-free manner, i.e., without permanent magnets. The rotor 10′ is then designed as a reluctance rotor, for example. Then, the magnetically effective core 101′ of the rotor 10′ is made of a soft magnetic material, for example. Suitable soft magnetic materials for the magnetically effective core 101′ are, for example, ferromagnetic or ferrimagnetic materials, i.e. in particular iron, nickel-iron, cobalt-iron, silicon-iron, mu-metal.

Furthermore, designs are possible in which the magnetically effective core 101′ of the rotor 10′ comprises both ferromagnetic materials and permanent magnetic materials. For example, permanent magnets can be placed or inserted into a ferromagnetic base body. Such designs are advantageous, for example, if one wishes to reduce the costs of large rotors by saving permanent magnetic material.

Typically, the magnetically effective core 101′ is completely encased in a plastic. In other designs, the magnetically effective core 101′ is completely enclosed in a sheathing 102′ consisting of a ceramic material or a metallic material, for example a stainless steel or titanium or tantalum.

Furthermore, the rotor 10′ comprises a plurality of vanes 103′ for conveying the fluid from the inlet 21′ to the outlet 22′.

The inlet 21′ of the pump housing 2′ is arranged and designed in such a way that the fluid to be conveyed flows towards the rotor 10′ in the axial direction A. The outlet 22′ extends parallel to the radial plane E, i.e. substantially perpendicular to the inlet 21′.

The pump housing 2′ comprises a cylindrical cup 31′ for receiving the rotor 10′. The cup 31′ is inserted into the recess 121′ in the stator housing 130′ so that the rotor 10′, more precisely the magnetically effective core 101′ of the rotor 10′, is arranged between the transverse legs 127′ of the coil cores 126′.

For many applications, for example for applications in the semiconductor industry, the pump unit 1′—with the exception of the magnetically effective core 101′—is made of a plastic, for example of a perfluoroalkoxy polymer (PFA) or of polytetrafluoroethylene (PTFE), because these are plastics with a particularly high chemical resistance. These plastics are practically inert materials that cannot be attacked even by chemically very aggressive substances, such as those frequently used in the semiconductor industry. In addition, PFA and PTFE are very pure plastics because they usually have no additives, and their molecular complexes are at least approximately inert. PFA is often preferred because it can be processed in injection molding processes.

In centrifugal pumps 200′, in which the fluid to be conveyed is redirected from the axial direction A into a radial direction, the rotor 10′ is subject to strong loads in the axial direction A. The axial thrust acting on the rotor is primarily caused by the pressure difference at the rotor 10′. While the suction pressure substantially prevails on the side of the rotor 10′ facing the inlet 21′, there is a higher pressure on the back side of the rotor 10′, because the back side of the rotor is connected to the outlet 22′, where the conveying pressure substantially prevails. The resulting axial thrust represents a challenge, particularly in centrifugal pumps 200′ with non-contact magnetically supported rotors 10′. In order to avoid that the axial thrust has to be received entirely by the axial magnetic bearing or stabilization of the rotor 10′, various measures are known, for example relief openings 104′ that extend in the axial direction A through the entire rotor 10′ and thus form a flow connection between the front side of the rotor 10′ facing the inlet 21′ and its back side, which leads to a pressure relief of the rotor 10′ with respect to the axial direction A.

For example, EP 2 273 124 proposes to divide the vanes 103′ of the rotor 10′ into two centrifugal wheels by a separating element 7′ aligned perpendicular to the axial direction A, namely a first centrifugal wheel 105′ for generating a main flow HF′ from the inlet 21′ to the outlet 22′, and a second centrifugal wheel 106′ for generating a recirculation flow RF′, which is directed from the back side of the rotor 10′ through the relief openings 104′. In FIG. 2, the main flow HF′ is indicated by solid arrows HF′, while the recirculation flow RF′ is indicated by dashed arrows RF′. Each vane 103′ is divided into a first vane 107′ and a second vane 108′ by the separating element 7′. The entirety of the first vanes 107′ forms the first centrifugal wheel 105′, and the entirety of the second vanes 108′ forms the second centrifugal wheel 108′. The first vanes 107′ are arranged in such a way that a central inlet area 25′ of the rotor 10′ is free of vanes 103′. The vanes 103′ are arranged around this central inlet area 25′.

The separating element 7′, which separates the two centrifugal wheels 105′ and 106′ from each other, redirects the recirculation flow RF′ from the axial direction A in the radial direction and at least partially separates the recirculation flow RF from the main flow HF, so that they cannot mix with each other directly at the exit of the relief openings 104′. In this case, the separating element 7′ extends with respect to the radial direction into the vanes 103′, i.e., in the radial direction, the separating element 7′ overlaps with the vanes 103′.

Even though this design with the separating element 7′ has proven itself in practice, the manufacture of such a rotor 10′ is very complex and elaborate. For example, it is necessary to assemble the rotor 10′ from several individual parts. For the separating element 7′, recesses must be provided in the vanes 103′ so that the separating element 7′ can be inserted between the vanes 103′.

If the pump unit 1′ is made of a plastic, for example, the individual components must be connected to each other in a reliable and stable manner. This takes place, for example, by welding processes. In addition to the time and cost factor, every welding process carries the risk of leaks occurring at the welded connection, whereby the operational reliability of the entire centrifugal pump 200′ is jeopardized. There is also the risk that cracks, or small gaps can occur at the welded connections. Contaminants can be deposited there, which then detach in the operating state and contaminate the fluid to be conveyed. In many applications, for example in the semiconductor industry, even the smallest impurities can have drastic consequences, for example, they can make the end product unusable.

To ensure that the components of the rotor 10′ that come into contact with the fluid, in particular the axial relief openings 104′ and also the inside located edges below the separating element 7′, can fulfill their intended function, an extremely high level of precision and dimensional accuracy of the components is necessary. In most cases, the components are manufactured using manufacturing processes (e.g. injection molding) in which deviations from a predetermined target geometry are unavoidable. This leads to the fact that protruding bulges or other deviations from the target geometry disturb the fluid flow and thus significantly impair the function. This means that it is necessary to rework the components before assembly, i.e. in their individual parts. If the components for rotor 10′ are already assembled, reworking is no longer possible because the areas of the rotor 10′ that require reworking are no longer sufficiently accessible for the corresponding reworking tools. This means, on the one hand, that the rotors 10′ known from the state of the art must be assembled from several individual parts and, on the other hand, that, apart from the assembly of the rotor 10′, additional work steps are necessary for the rotor 10′ to fulfill its intended functions.

Starting from this state of the art, it is therefore an object of the disclosure to propose a pump unit with a rotor for a centrifugal pump that can be magnetically levitated without contact, which pump unit is particularly simple with regard to its manufacture and is characterized by a high degree of operational reliability. In addition, it is an object of the disclosure to propose a centrifugal pump with such a pump unit.

The subject matter of the disclosure meeting this object is characterized by the features disclosed herein.

According to the disclosure, a pump unit for a centrifugal pump is thus proposed, which comprises the pump unit and a stator extending in an axial direction from a first axial end to a second axial end, wherein a cup-shaped recess is provided at the first axial end, into which the pump unit can be inserted, wherein the pump unit has a pump housing with an inlet and with an outlet for a fluid to be conveyed as well as a rotor arranged in the pump housing with a plurality of vanes for conveying the fluid, wherein the rotor can be rotated about the axial direction, wherein the pump unit is designed for a non-contact magnetic levitation of the rotor and for a non-contact magnetic drive of the rotor by the stator, wherein the vanes of the rotor are arranged around a central inlet area of the rotor, wherein the rotor comprises at least one relief opening for generating a recirculation flow which is directed from a back side of the rotor facing away from the inlet in the direction of the central inlet area of the rotor, and wherein the rotor further has a separating element arranged in the central inlet area, which redirects the recirculation flow in a radial direction perpendicular to the axial direction. The plurality of vanes, the separating element and the at least one relief opening of the rotor are designed as a one-piece unit.

Due to the one-piece design of the unit with the vanes, the separating element and the at least one relief opening of the rotor, the rotor no longer has to be assembled from several components but can be manufactured as a monolithic device in a very simple manner. In addition, it does not require any connections between individual components, for example by gluing, screwing or welding, whereby, on the one hand, the constructive effort is reduced, and, on the other hand, operational safety is increased because, for example, welded connections are no longer necessary, which could lead to leakages in the operating state.

For example, it is possible to design the one-piece unit comprising the vanes, the separating element and the at least one relief opening as a one-piece injection-molded part. The manufacture in an injection molding process enables a particularly cost-effective and economical manufacture of the rotor. In addition, the rotor is then necessarily designed such that it can be demolded, i.e. it can be removed from the tool after the injection molding process.

According to a preferred embodiment, exactly one relief opening is provided, which connects the central inlet area of the rotor to the back side of the rotor. This relief opening is then centrally arranged in the rotor.

In other embodiments of the pump unit according to the disclosure, several relief openings are provided, which are arranged around a center axis of the rotor, wherein each relief opening connects the central inlet area of the rotor to the back side of the rotor. For example, the relief openings are arranged on a circular line whose center lies on the center axis of the rotor. In these embodiments, a relief opening can also be provided in the center of the rotor, which encloses the center axis. The other relief openings are then arranged around the relief opening in the center.

Preferably, the rotor comprises a ring-shaped or disk-shaped magnetically effective core, as well as a sheathing which completely encloses the magnetically effective core, and wherein the sheathing is a component of the one-piece unit, which comprises the vanes and the separating element. In this embodiment, the sheathing, the vanes, the separating element and all relief openings are designed as a monolithic component.

In a preferred embodiment, the separating element is designed and arranged in such a way that the at least one relief opening is partially visible from the inlet. This means that the separating element does not completely cover the relief opening or relief openings. This has the advantage that the relief opening(s) is (are) accessible from the pump inlet, whereby, for example, a machining of the relief opening(s), for example, a chip-removing subsequent machining, is made possible.

According to a preferred embodiment, the separating element comprises a separating plate and attachment webs, wherein the separating plate has a maximum outer diameter in the radial direction, which is at most as large as the diameter of the central inlet area of the rotor, and wherein the attachment webs are designed to fix the separating plate. Due to the design with the attachment webs, it is no longer necessary, but still possible, to attach the separating element to the vanes, whereby the constructive effort is reduced.

Preferably, the separating plate is designed such that the maximum outer diameter of the separating plate is smaller than the diameter of the central inlet area of the rotor. Then, the separating plate is dimensioned with respect to the radial direction such that it can be arranged between the vanes without touching the vanes.

It is a further preferred embodiment that each attachment web extends from the separating plate to the sheathing, wherein a radial opening for the recirculation flow is provided in each case between adjacent attachment webs. In this way, the separating plate is fixed to the sheathing, wherein the recirculation flow can flow out between the attachment webs in the radial direction. Here, it is preferred that the radial openings between the attachment webs are arranged such that they are aligned with the interspaces between two adjacent vanes when viewed in the radial direction, so that the recirculation flow can flow unhindered between two adjacent vanes.

It is a preferred embodiment that each attachment web extends from a lower side of the separating plate in the axial direction to the sheathing. In this embodiment, the attachment webs are preferably completely covered by the separating plate so that the separating webs are not visible from the inlet.

According to another preferred embodiment, each attachment web is arranged at the outer edge of the separating plate and extends from the outer edge in the radial direction. In this embodiment of the attachment webs as radial struts, the attachment webs are visible from the inlet. When viewed from the inlet of the pump housing, the separating element then looks star-shaped.

In those embodiments in which the attachment webs are arranged at the outer edge of the separating plate, it is preferred that the attachment webs are arranged equidistantly on the outer edge of the separating plate.

It is a preferred variant of this embodiment that each attachment web extends in each case in the radial direction to one of the vanes. Each of the attachment webs is then in direct physical contact with one of the vanes. This embodiment also has the advantage that the radial openings for the recirculation flow, which are arranged between the attachment webs, merge into the radial openings between adjacent vanes, whereby in particular turbulence can be avoided or at least drastically reduced.

Furthermore, it is preferred, especially for these embodiments, that the number of attachment webs be equal to the number of vanes. In this way, continuous channels are formed for the recirculation flow.

Furthermore, a centrifugal pump for conveying a fluid is proposed by the disclosure, with a pump unit which is designed according to any one of the preceding aspects, and which has a cylindrical cup for receiving the rotor, as well as with a stator extending in an axial direction from a first axial end to a second axial end, wherein a cup-shaped recess is provided at the first axial end, into which the cylindrical cup of the pump unit can be inserted, wherein the stator together with the rotor forms an electromagnetic rotary drive for rotating the rotor about the axial direction, wherein the stator is designed as a bearing and drive stator with which the rotor can be magnetically driven without contact and magnetically levitated without contact with respect to the stator, wherein the rotor is passively magnetically stabilized with respect to the axial direction and is actively magnetically levitated in a radial plane perpendicular to the axial direction.

Particularly preferably, the electromagnetic rotary drive is designed as a temple motor, wherein the stator has a plurality of coil cores, each of which comprises a longitudinal leg extending from a first end in the axial direction to a second end, as well as a transverse leg which is arranged at the second end of the longitudinal leg and in the radial plane, and which extends from the longitudinal leg in the radial direction, wherein the coil cores are arranged around the rotor with respect to the circumferential direction, so that the rotor is arranged between the transverse legs of the coil cores, and wherein at least one concentrated winding is provided at each longitudinal leg, which winding surrounds the respective longitudinal leg.

Further advantageous measures and embodiments of the disclosure are apparent from the disclosure.

### BRIEF DESCRIPTION OF THE DRAWINGS

In the following, the disclosure will be explained in more detail with reference to embodiments and with reference to the drawing. In the schematic drawing show (partially in section):

FIG. 1 is a perspective view of a centrifugal pump according to the state of the art, partially in section,

FIG. 2 is a pump unit according to the state of the art in a sectional view,

FIG. 3 is an embodiment of a pump unit according to the disclosure in a sectional view,

FIG. 4 is a perspective view of the rotor of the pump unit from FIG. 3, partially in section,

FIG. 5 is a sectional view of the rotor in a section along the section line V-V in FIG. 4,

FIG. 6 is a first variant of the rotor in a sectional view,

FIG. 7 is a perspective view of the first variant of the rotor from FIG. 6, partially in section,

FIG. 8 is a sectional view of the first variant of the rotor in a section along the section line VIII-VIII in FIG. 7,

FIG. 9A is a second variant of the rotor in a sectional view,

FIG. 9B is the second variant of the rotor in a plan view from the inlet of the pump housing,

FIG. 10 is a perspective view of the second variant of the rotor from FIG. 9A, partially in section,

FIG. 11 is a sectional view of the second variant of the rotor in a section along the section line XI-XI in FIG. 10,

FIG. 12 is a third variant of the rotor in a plan view from the inlet of the pump housing,

FIG. 13 is a perspective view of the third variant of the rotor from FIG. 12, partially in section,

FIG. 14 is a sectional view of the third variant of the rotor in a section along the section line XIV-XIV in FIG. 13, and

FIG. 15 is a schematic sectional view of an embodiment of a centrifugal pump according to the disclosure.

### DETAILED DESCRIPTION

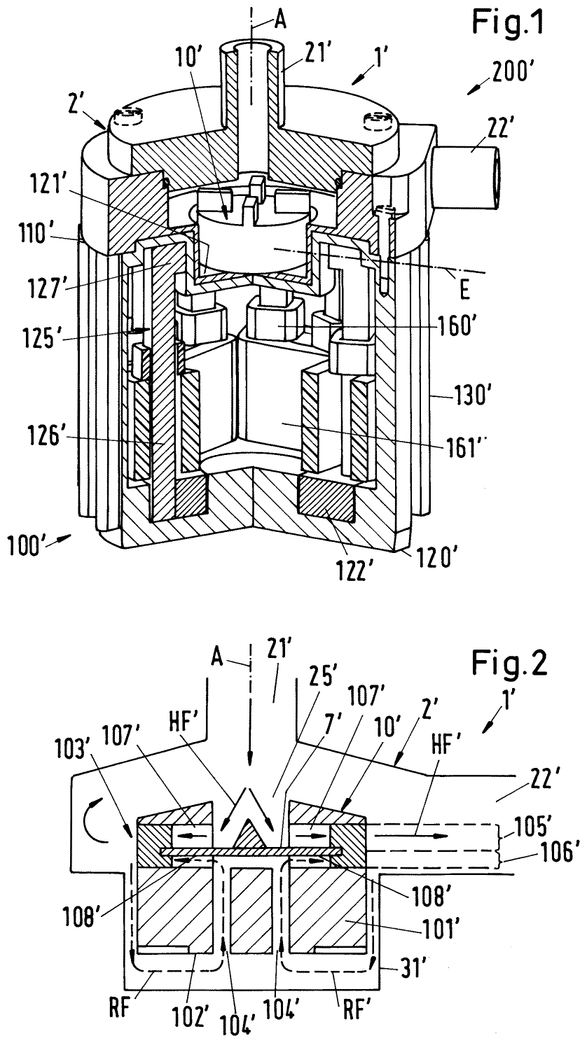

As already explained above, FIG. 1 shows a centrifugal pump 200′ with a non-contact magnetically supported and non-contact magnetically driven rotor 10′, which is known from the state of the art. In a sectional view, FIG. 2 shows a pump unit 1′, which is known from the state of the art, and which is suitable for the centrifugal pump 200′ from FIG. 1, for example.

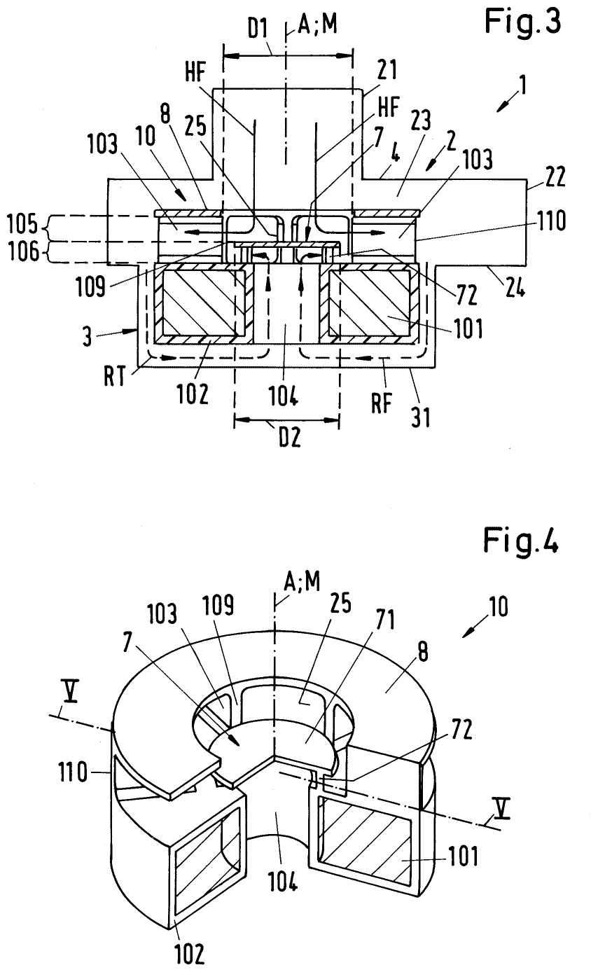

FIG. 3 shows in a sectional view corresponding to FIG. 2 an embodiment of a pump unit according to the disclosure, which is designated in its entirety with the reference sign 1.

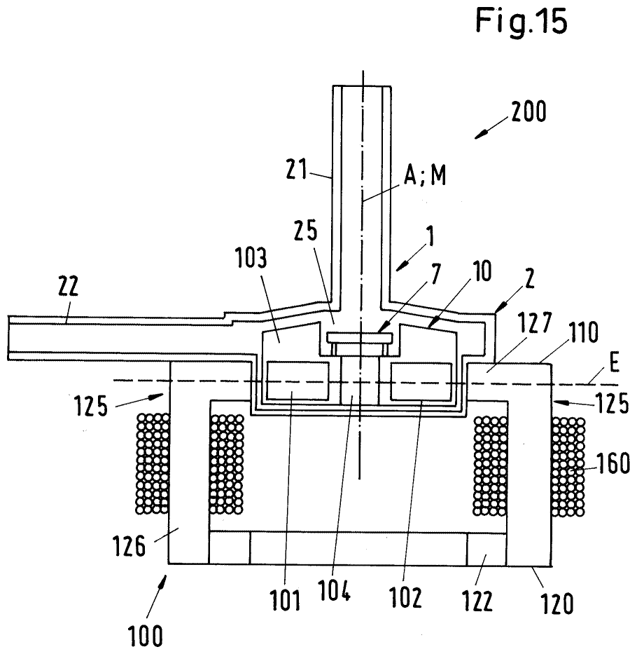

The pump unit 1 is designed for a centrifugal pump 200 (see FIG. 15) for conveying a fluid and comprises a pump housing 2 with an inlet 21 and with an outlet 22 for the fluid. A rotor 10 for conveying the fluid is arranged in the pump housing 2, which rotor forms the centrifugal wheel or the impeller of the pump unit 1 and thus of the centrifugal pump 200. The rotor 10 can be rotated about a desired axis of rotation, which defines an axial direction A. This desired axis of rotation is the center axis M of the rotor 10.

With respect to the axial direction A, the rotor 10 extends from a front side facing the inlet to a back side facing away from the inlet 21.

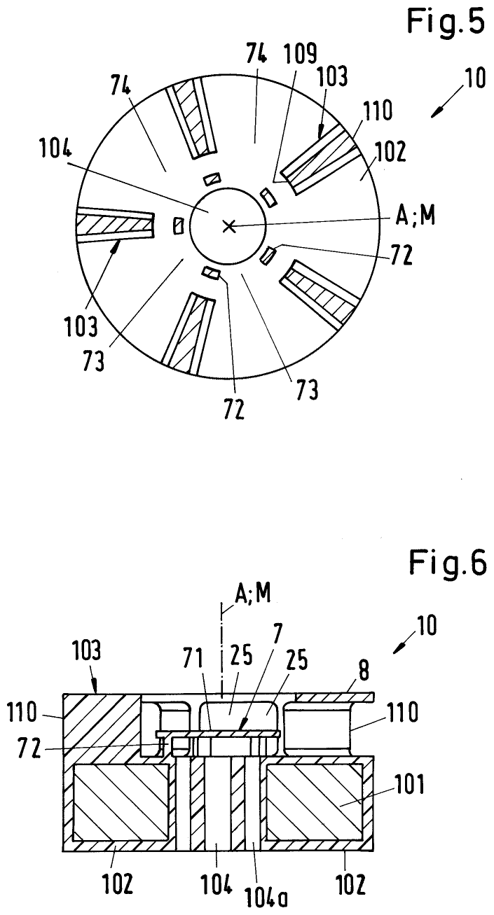

For better understanding, FIG. 4 shows the rotor 10 of the pump unit 1 in a perspective view, wherein a sector is cut out of the rotor 10. Furthermore, FIG. 5 shows the rotor 10 in a sectional view. The section is made along the section line V-V in FIG. 4.

A direction perpendicular to the axial direction A is designated as the radial direction. In the following, the term “axial” is used with the generally accepted meaning “in the axial direction” or “with respect to the axial direction”. The term “radial” is used with the generally accepted meaning “in the radial direction” or “with respect to the radial direction”.

The pump unit 1 is designed for a non-contact magnetic levitation of the rotor 10 and for a non-contact magnetic drive of the rotor 10. This can be realized in particular in the analogously same way as explained on the basis of FIG. 1 and FIG. 2. Thus, the pump unit 1 according to the disclosure can be designed in the analogously same way with respect to the magnetic levitation and the magnetic drive as the pump unit 1′ in FIG. 1 or in FIG. 2. For this purpose, the rotor 10 of the pump unit 1 comprises a magnetically effective core 101, which is designed, for example, as a permanent magnetic ring or permanent magnetic disk and is enclosed by a sheathing 102. The sheathing 102 is preferably designed as a plastic sheathing. The sheathing 102 is made of PTFE or PFA, for example. The magnetically effective core 101 is encapsulated in the sheathing 102, i.e. the sheathing 102 completely and preferably hermetically encloses the magnetically effective core 101. As a result, the magnetically effective core 101 is protected from the fluid. The sheathing 102 can be manufactured, for example, by spraying a plastic around the magnetically effective core 101.

The magnetically effective core 101 of the rotor 10 is that component of the rotor 10, which cooperates magnetically with the stator 100 to generate the torque as well as to generate the magnetic levitation forces.

Furthermore, the rotor 10 comprises a plurality of vanes 103 for conveying the fluid from the inlet 21 to the outlet 22. The vanes 103 are arranged on the sheathing 102 of the magnetically effective core 101. The vanes 103 are preferably made of plastic and are preferably designed in one piece with the sheathing 102. Of course, it is also possible to manufacture the individual vanes 103 or the entirety of the vanes 103 in a separate manufacturing process and then connect them to the sheathing 102 of the magnetically effective core 101, for example by a welding process.

The impeller with the vanes 103 formed by the rotor 10 is preferably designed as radial impeller, which is approached by the fluid from the inlet 21 in the axial direction A, and then redirects the fluid in a radial direction.

The pump housing 2 comprises a cover part 4 and a bottom part 3, which are connected to each other in a sealing manner, which is not represented in more detail in FIG. 3 because it is not necessary for the understanding of the disclosure. The bottom part 3 comprises a cylindrical cup 31 for receiving the rotor 10. The cup 31 is preferably designed and arranged such that it can be inserted into a cup-shaped recess of a stator 100, as is represented schematically in FIG. 15.

The stator 100 (FIG. 15) extends in the axial direction A from a first axial end 110 to a second axial end 120 and has a stator housing (not represented in FIG. 15), which is substantially designed in a cylindrical manner. The cup-shaped recess is arranged at the first axial end 110 of the stator 100, preferably centrally in the end face which forms the first axial end 110 of the stator 100. The design of the stator housing with the cup-shaped recess can in particular be realized in a way analogous to that explained on the basis of FIG. 1 for the stator housing 130′ and the cup-shaped recess 121′. Thus, the cup 31 is then arranged and designed in such a way that it can be inserted into the recess 121′ (FIG. 1) in the first axial end 110′ of the stator 100′, and the magnetically effective core 101 is arranged between the transverse legs 127′ of the coil cores 125′.

As can be recognized in FIG. 3, there is a pump chamber 23 above the cup 31 according to the representation, which is delimited by the pump housing 2 and in which the vanes 103 of the rotor 10 are arranged. The pump chamber 23 is designed at least substantially in a cylindrical manner, wherein the diameter of the pump chamber 23 is larger than the inner diameter of the cup 31. As a result, the pump housing 2 has a flange-like projection 24, which delimits the pump chamber 23 downward with respect to the axial direction A according to the representation.

The inlet 21 is centrally arranged in the cover part 4 of the pump housing 2 so that the fluid can flow towards the rotor 10 in the axial direction A.

Each vane 103 extends from a radially inwardly arranged leading edge 109 to a radially outwardly arranged trailing edge 110. In the embodiment described here, the vanes 103 are designed by way of example such that they extend in a straight line in the radial direction from the leading edge 109 to the trailing edge 110 and have a constant height over their entire extension. Here, the height refers to the extension of the vanes 103 in the axial direction A. As mentioned, this design is only to be understood as an example. In other embodiments, the vanes 103 are designed, for example, in a curved manner with respect to the radial direction, and/or with a height in the axial direction A which changes from the leading edge 109 to the trailing edge 110. For example, an embodiment is shown in FIG. 15 in which the vanes 103 have a greater height at their leading edge 109 than at their trailing edge 110.

Furthermore, a ring-shaped cover plate 8 is provided, which is arranged on the upper edges of the vanes 103 facing the inlet 21. The cover plate 8 covers all vanes 103. With respect to the radial direction, the ring-shaped cover plate 8 extends from the leading edges 109 of the vanes 103 to their trailing edges 110. The cover plate 8 can designed in one piece with the vanes 103.

As can be particularly clearly recognized in FIG. 4 and FIG. 5, the vanes 103 of the rotor 10 are arranged around a central inlet area 25 which is free of vanes 103. The leading edges 109 of the vanes 103 lie on a line, here a circular line, which has a distance from the center axis M of the rotor 10 that is different from zero. The diameter D1 of the central inlet area 25 is determined by the distance of the leading edges 109 of the vanes 103 from the center axis M of the rotor 10. This diameter D1 of the central inlet area 25 is equal to twice the distance of the leading edges 109 of the vanes 103 from the center axis of the rotor 10. In the embodiment described here, the diameter D1 of the central inlet area 25 is the same size as the inner diameter of the ring-shaped cover plate 8.

If the leading edges 109 of the vanes run not parallel to the axial direction A but are inclined with respect to the axial direction A, for example, the diameter D1 of the central inlet area 25 is determined by the distance of the leading edges 109 at the upper edges of the vanes 103 facing the inlet 21.

The rotor 10 further comprises at least one relief opening 104 for generating a recirculation flow RF which is directed from the back side of the rotor 10 facing away from the inlet 21 in the direction of the central inlet area 25. In the embodiment represented in FIG. 3 to FIG. 5, exactly one relief opening 104 is provided, which is designed in a circular cylindrical manner and extends in the axial direction A. The relief opening 104 is arranged centrally in the rotor 10, such that the axis of this relief opening 104 coincides with the center axis M of the rotor 10. The relief opening 104 extends from the central inlet area 25 in the axial direction A through the rotor 10 to the back side of the rotor 10. The rotor 10 further comprises a separating element 7, which is arranged in the central inlet area 25 of the rotor 10, and which redirects the recirculation flow RF. which flows from the back side of the rotor 10 through the relief opening 104, from the axial direction A into the radial direction.

Preferably, the separating element 7 comprises a separating plate 71, which is aligned perpendicular to the axial direction A, and a plurality of attachment webs 72 for fixing the separating plate 71. The separating plate 71 is designed here in the form of a circular disk and has an outer diameter D2. In other embodiments, the separating plate 71 can also have a shape that differs from a circular disk and/or can have flow directing elements. In this case, the outer diameter D2 refers to the maximum outer diameter D2, i.e. the maximum extension of the separating plate 71 in the radial direction.

The separating plate 71 is arranged in the central inlet area 25 centered with respect to the radial direction, i.e., the center point of the separating plate 71 lies on the center axis M of the rotor 10. With respect to the axial direction A, the separating plate 71 is arranged such that for each vane 103, an upper part of the leading edge 109 is arranged above the separating plate 71 with respect to the axial direction A, according to the representation (FIG. 3, FIG. 4), and a lower part of the leading edge 109 is arranged below the separating plate 71 with respect to the axial direction A.

The entirety of the areas of the vanes 103 arranged above the separating plate 71 with respect to the axial direction A form a first centrifugal wheel 105, which primarily serves to generate the main flow HF, which flows from the axial direction A from the inlet 21 to the outlet 22. The main flow HF is indicated by the arrows HF represented by solid lines.

The entirety of the areas of the vanes 103 arranged below the separating plate 71 with respect to the axial direction A form a second centrifugal wheel 106, which primarily serves to generate the recirculation flow RF, which is directed from the back side of the rotor 10, facing away from the inlet 21, through the relief opening 104 in the direction of the central inlet area 25. The recirculation flow RF is indicated by the arrows RF represented by dashed lines.

The separating element 7 redirects the recirculation flow RF from the axial direction A in the radial direction. The separating element 7 prevents a direct collision or a direct contact of the main flow HF with the recirculation flow RF in the area of that end of the relief opening 104 that faces the central inlet area 25. Due to the separating element 7, it is thus prevented that the recirculation flow RF and the main flow HF meet head-on, i.e. as flows directed opposite to each other. Due to the separating element 7, the recirculation flow RF is thus at least partially separated from the main flow HF in the area of the central inlet area 25. The recirculation flow RF is initially redirected in the radial direction by the separating element 7. A substantial mixing of the main flow HF and the recirculation flow RF does not occur until the recirculation flow RF passes the outer edge of the separating plate 71. The outer edge of the separating plate 71 refers to the radially outer edge of the separating plate 71. Since the recirculation flow RF has already been redirected in the radial direction, it can mix with the main flow HF almost free of strong turbulence.

As can be recognized best in FIG. 3, the outer diameter D2 of the separating plate 71 is smaller than the diameter D1 of the central inlet area 25. In this embodiment, the separating plate 71 is located entirely in the central inlet area 25 and has no physical contact with the vanes 103. The leading edges 109 of the vanes 103 are arranged around the separating plate 71 without touching it. This embodiment has the advantage that the separating plate 71 can be machined in a simple way, for example during the manufacture of the rotor 10.

The outer diameter D2 of the separating plate 71 is larger than the inner diameter of the relief opening 104, so that the relief opening 104 is completely covered by the separating plate 71. Thus, the relief opening 104 is not visible from the inlet 21.

However, such embodiments are also possible in which the relief opening 104 is partially visible from the inlet 21, i.e. in which the separating plate 71 does not completely cover the relief opening 104. For example, this can be realized in such a way that the outer diameter D2 of the separating plate 71 is smaller than the inner diameter of the relief opening 104.

The separating element 7 comprises the attachment webs 72 for fixing the separating plate 71. All attachment webs 72 are arranged at the lower side of the separating plate 71. Here, the lower side of the separating plate 71 refers to that boundary surface of the separating plate 71 which faces the relief opening 104. Each attachment web 72 extends from the lower side of the separating plate 71 in axial direction A to the sheathing 102, on which the attachment web 72 is supported. The separating plate 71 is thus fixed to the sheathing 102 by the attachment webs 72. Therefore, it is no longer necessary to attach the separating plate 71 or the separating element 7 to the vanes 103.

As can be recognized best in FIG. 5, the attachment webs 72 are arranged on a circle with respect to the radial direction, which is concentric with the relief opening 104 and has a larger diameter than the relief opening 104. The diameter of the circle on which the attachment webs are arranged, is smaller than the outer diameter D2 of the separating plate 71. The attachment webs 72 are thus arranged at the lower side of the separating plate 71 in such a way that they are not visible from the inlet 21 of the pump housing 2.

The attachment webs 72 are preferably arranged equidistantly around the relief opening 104. A total of five attachment webs 72 are provided. Between two adjacent attachment webs, a radial opening 73 is provided in each case, through which the recirculation flow RF can flow out of the relief opening 104 in the radial direction towards the outlet 22.

Preferably, the attachment webs 72 are arranged such that the radial openings 73 are aligned in the radial direction with the interspaces 74 between the leading edges 109 of two adjacent vanes 103. This can be recognized best in FIG. 5. Thus, the recirculation flow RF flowing out of the radial openings 73 can flow unhindered into the interspaces 74 between adjacent vanes 103. In this way, the formations of vortices in the recirculation flow RF are at least significantly reduced. It is particularly advantageous for this embodiment, if the number of attachment webs 72 is equal to the number of vanes. In the embodiment described here, the rotor 10 has exactly five vanes by way of example. Accordingly, the separating element 7 has exactly five attachment webs 72, each of which is located on a connecting line between the center axis M of the rotor 10 and one of the leading edges 109 of the vanes 103. Thus, each of the five radial openings 73 is aligned in each case in the radial direction with exactly one of the interspaces 74 between adjacent vanes 103.

According to the disclosure, the plurality of vanes 103, the separating element 7 and the relief opening 104 are designed as a one-piece unit. Particularly preferably, the sheathing 102 is also a component of this one-piece unit. Furthermore, it is preferred that the cover plate 8, which is arranged on the vanes 103, is also a component of this one-piece unit. Particularly preferably, the rotor 10 as a whole, with the exception of the magnetically effective core 101, is designed as a one-piece unit. In this embodiment, it is thus no longer necessary to connect the individual components of the rotor 10 with one another by joining methods such as gluing, welding, screwing or similar. The one-piece unit has a monolithic design, i.e. it is not composed of several components, but it is a single piece. Consequently, the one-piece unit is free of adhesions, screw connections, welding seams, seals and contacts between adjacent components.

Due to the one-piece design of the unit, which comprises at least the vanes 103, the separating element 7 and the at least one relief opening 104, and preferably all components of the rotor 10 with the exception of the magnetically effective core 101, the rotor 10 no longer has to be assembled from several components but can be designed as a monolithic device.

Since there is no need for individual components to be joined by gluing, screwing or welding, for example, and since no seals are required between individual components of the rotor 10, this results in very high operational reliability.

The one-piece unit, for example, is designed as a one-piece injection-molded part, i.e. it can be manufactured by an injection molding process. The injection molding process is preferably designed such that the magnetically effective core 101 is integrated into the injection molding process. The magnetically effective core 101 can, for example, be sprayed with a plastic in the injection molding process to manufacture the sheathing 102 in this way.

Preferably, the rotor 10 is manufactured by combining an injection molding process with a subsequent subtractive machining method, for example a chip-removing machining method such as milling or drilling.

Of course, other methods are also suitable for manufacturing the rotor 10, for example, additive manufacturing methods such as the method designated as 3D printing.

Preferably, the one-piece unit includes or consists of a plastic. For example, the one-piece unit can be injection molded from one of the following plastics:

Polyvinyl chloride (PVC), perfluoralkoxy polymers (PFA), polypropylene (PP), polyethylene (PE).

It is also possible to manufacture the rotor 10 by a sintering process and a subsequent subtractive machining method. The sheathing 102 is then made, for example, of a powder or a granulate that is pressed onto the magnetically effective core 101 using pressure and, optionally, a heat treatment, in such a way that the magnetically effective core 101 is completely enclosed. Applying heat and/or pressure, the plastic is molded around the magnetically effective core 101 to form a monolithic block, for example, a cylindrical body. Then, the rotor 10 with the vanes 103, the separating element 7, the at least one relief opening 104 and optionally the cover plate 8 is brought into the desired shape by a chip-removing machining.

Furthermore, it is possible, in addition to or instead of a powder or a granulate, to join several plastic parts into a monolithic block using heat and/or pressure, in which the magnet has been inserted in advance and which completely encloses the magnet after the joining process. Then, the rotor 10 with the vanes 103, the separating element 7, the at least one relief opening 104 and optionally the cover plate 8 is brought into the desired shape by a chip-removing machining.

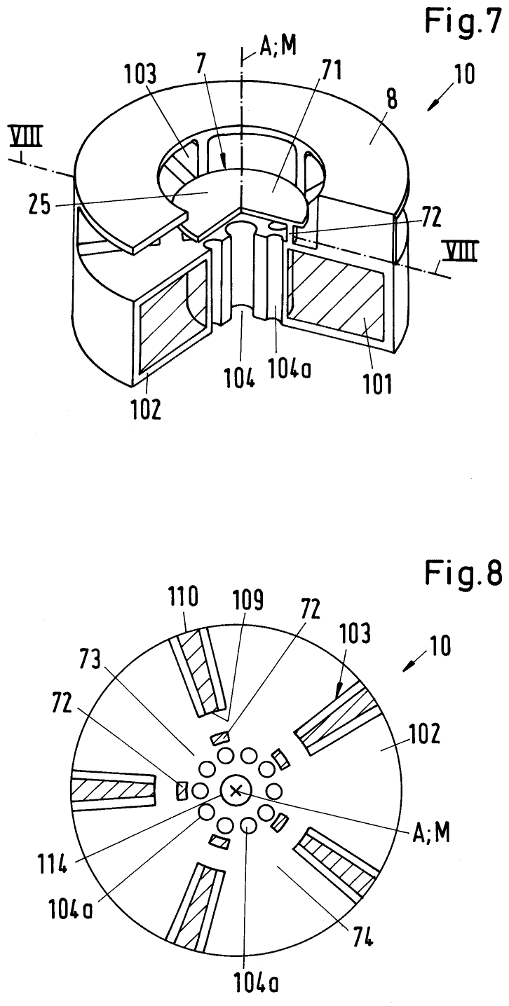

In FIG. 6 to FIG. 8, a first variant of the rotor 10 is represented. FIG. 6 shows the rotor in a sectional view, wherein the section is made along the axial direction A. FIG. 7 shows a perspective view, partially in section, which corresponds to the view in FIG. 4. FIG. 8 shows a sectional view of the first variant of the rotor 10 along the section line VIII-VIII in FIG. 7. The view in FIG. 8 corresponds to that in FIG. 5.

In the first variant of the rotor 10, several relief openings 104, 104a are provided. One of the relief openings 104 is again arranged centrally in the rotor 10, so that the axis of this relief opening 104 coincides with the center axis M of the rotor 10. A plurality of additional relief openings 104a is arranged around this centrally arranged relief opening 104. By way of example, ten additional relief openings 104a are provided here, which are arranged on a circle whose center point is on the center axis of the rotor 10. Each of the relief openings 104, 104a is designed in each case as a cylindrical bore or opening which extends from the central inlet area 25 in the axial direction A through the rotor 10 to its back side. All relief openings 104, 104a are arranged parallel to one another. The circle on which the additional relief openings 104a are arranged has a diameter which is smaller than the outer diameter D2 of the separating plate 71, such that all relief openings 104, 104a are completely covered by the separating plate 71. Thus, none of the relief openings 104, 104a is visible from the inlet 21.

However, such embodiments are also possible in which one or more of the relief openings 104, 104a are partially or completely visible from the inlet 21, i.e. where the separating plate 71 does not completely cover all the relief openings 104, 104a. For example, this can be realized such that the outer diameter D2 of the separating plate 71 is the same size as or smaller than the diameter of the circle on which the additional relief openings 104a are arranged.

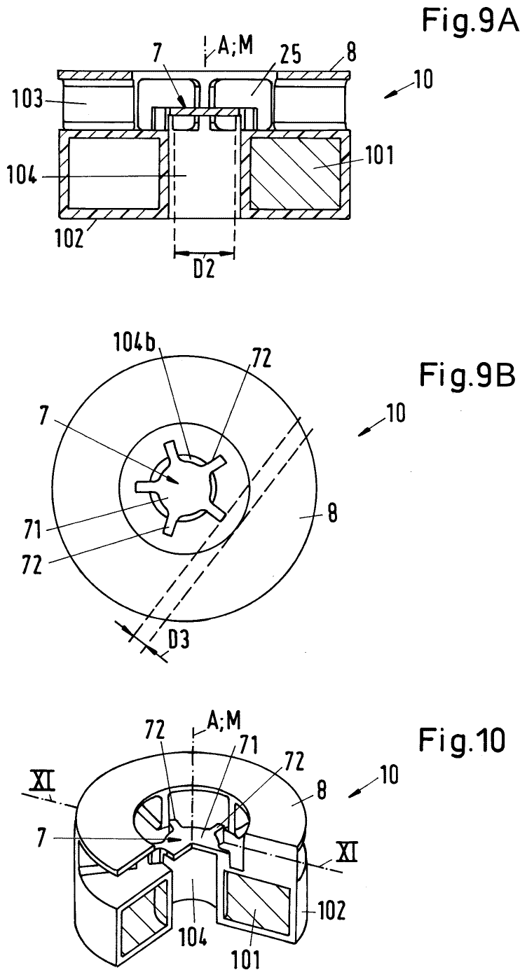

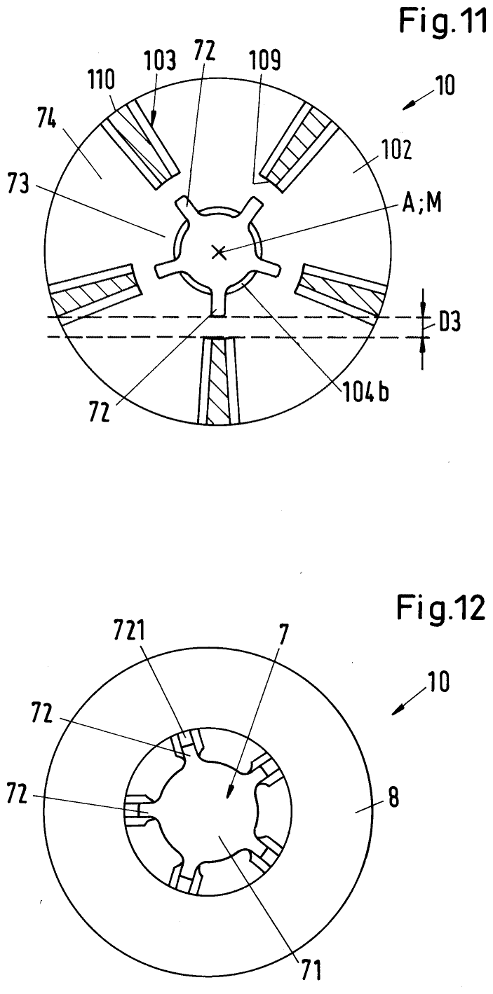

In FIG. 9A, FIG. 9B, FIG. 10 and FIG. 11, a second variant of the rotor 10 is represented. FIG. 9A shows the rotor 10 in a sectional view, wherein the section is made along the axial direction A. FIG. 9B shows the rotor 10 in a plan view from the inlet 21 of the pump housing 2. FIG. 10 shows a perspective view, partially in section, corresponding to the representation in FIG. 4. FIG. 11 shows a sectional view of the second variant of the rotor 10 along the section line XI-XI in FIG. 10. The view in FIG. 11 corresponds to that in FIG. 5. In the second variant of the rotor 10, again only one relief opening 104 is provided, which is arranged centrally. It is understood that such embodiments of the second variant of the rotor 10 are also possible, in which several relief openings 104, 104a are provided, for example in the analogously same way as described for the first variant of the rotor 10 (see FIG. 8).

In the second variant of the rotor 10, the attachment webs 71 of the separating element 7, with which the separating plate 71 is fixed, are arranged at the outer edge of the separating plate 71. Each attachment web 72 extends from the outer edge of the separating plate 7 in a radial direction outwards. In addition, each attachment web 71 also extends in the axial direction A to the sheathing 102, on which the attachment web 71 is supported. In this embodiment with the attachment webs 72 arranged at the outer edge of the separating plate 71, it is also preferred that the number of attachment webs 72, here for example five, is the same as the number of vanes 103 of the rotor 10. When viewed from the inlet, the separating element 7, with the separating plate 71 and the attachment webs 72 arranged at its edge, has a star-shaped appearance.

The attachment webs 72 are preferably arranged equidistantly at the outer edge of the separating plate 71. Between two adjacent attachment webs 72, one of the radial openings 73 is arranged in each case, through which the recirculation flow RF can flow from the relief opening 104 in the radial direction towards the outlet 22.

Preferably, the attachment webs 72 are also arranged in the second variant of the rotor 10 such that the radial openings 73 are aligned in the radial direction with the interspaces 74 between the leading edges 109 of two adjacent vanes 103. This can be recognized best in FIG. 11. Thus, the recirculation flow RF flowing out of the radial openings 73 can flow unhindered into the interspaces 74 between adjacent vanes 103. In this way, the formations of vortices in the recirculation flow RF are at least significantly reduced.

In the second variant of the rotor 10, the separating plate 71 of the separating element 10 is designed again in the shape of a circular disk. As can be recognized in FIG. 9A, in the second variant of the rotor 10, the outer diameter D2 of the separating plate 71 is smaller than the inner diameter of the centrally arranged relief opening 104. As a result, the relief opening 104 is partially visible from the inlet 21, because the separating plate 71 does not completely cover the relief opening 104. There is a ring-shaped gap 104b around the separating plate 71, which is not covered by the separating plate 71 and is visible from the inlet 21 of the pump housing 2. This embodiment has the advantage that during the manufacture of the rotor 10, a milling tool can plunge into the ring-shaped gap 104b in the axial direction A, whereby in particular the separating plate 71 can be more easily or more precisely machined in the area between two adjacent attachment webs 72.

Furthermore, it is preferred that the attachment webs 72 end with respect to the radial direction at a distance D3 in front of the leading edges 109 of the vanes 103, wherein D3 is sufficiently large so that a milling tool can fit between the attachment webs 72 and the leading edges 109 of the vanes 103. D3 is therefore the distance, measured in the radial direction, between the radially outer end of the attachment webs 72 and the leading edges 109 of the vanes 103. In the embodiment represented in FIG. 11, the distance D3 is equal to the distance, measured in the radial direction, between the radially outer ends of the attachment webs 72 and the radially inside located edge of the cover plate 8. From a practical point of view, it is preferred that the distance D3 is at least as large as one thirtieth, preferably at least as large as one fifteenth, of the distance measured in the axial direction A between the upper side of the sheathing 102, on which the vanes 103 are arranged, and the upper side of the rotor 10 facing the inlet 21 at the inlet area 25, which here is formed by the cover plate 8. Of course, such embodiments of the rotor are also possible in the second variant in which the relief opening 104 is completely covered by the separating plate 71, so that the relief opening 104 is not visible from the inlet 21. For this purpose, for example, the outer diameter D2 of the separating plate 71 is larger than the inner diameter of the relief opening 104.

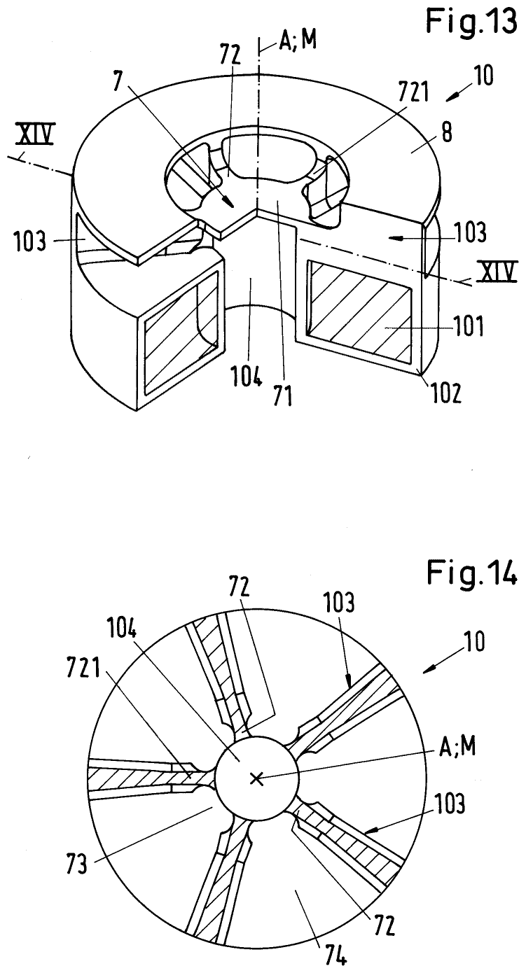

A third variant of the rotor 10 is represented in FIG. 12 to FIG. 14. FIG. 12 shows the rotor 10 in a plan view from the inlet of the pump housing 2. FIG. 13 shows a perspective view, partially in section, which corresponds to the representation in FIG. 4. FIG. 14 shows a sectional view of the third variant of the rotor 10 along the section line XIV-XIV in FIG. 13. The representation in FIG. 14 corresponds to that in FIG. 5.

The third variant of the rotor 10 is designed in a similar way to the second variant and in particular with the attachment webs 72, which are arranged at the outer edge of the separating plate 71. However, the attachment webs 72 extend in the radial direction up to the vanes 103. Each attachment web 72 thus extends in each case in the radial direction to the leading edge 109 of one of the vanes 103. Preferably, each of the attachment webs 72 merges in each case into one of the vanes 103. Preferably, a transition area 721, which is deigned in a rounded manner, is provided at the radially outer end of each attachment web 72, in which transition area the attachment web 72 merges into the leading edge 109 of the vane 103.

Furthermore, the centrifugal pump 200 for conveying a fluid with a pump unit 1 is proposed by the disclosure, wherein the pump unit 1 is designed according to the disclosure. In a schematic sectional view, FIG. 15 shows an embodiment of a centrifugal pump 200 according to the disclosure. The centrifugal pump 200 comprises the stator 100, which extends in the axial direction A from a first axial end 110 to a second axial end 120, wherein a cup-shaped recess (not represented in FIG. 15) is provided at the first axial end 110, into which the cylindrical cup 31 of the pump unit 1 can be inserted. The stator 100 together with the rotor 10 forms an electromagnetic rotary drive for rotating the rotor 10 about the axial direction A, wherein the stator 100 is designed as a bearing and drive stator with which the rotor 10 can be driven magnetically without contact and can be magnetically levitated without contact with respect to the stator 100, wherein the rotor 10 is passively magnetically stabilized with respect to the axial direction A and is actively magnetically levitated in a radial plane E perpendicular to the axial direction A.

The stator 100 comprises a stator housing, which is not represented in FIG. 15 for reasons of a better overview. However, the stator 100 can, for example, be designed in the analogous way as the stator 100′ with the stator housing 130′ represented in FIG. 1, whereby the recess 121′ is provided in the stator housing 130′, into which the cylindrical cup 31 of the bottom part 3 of the pump housing 1 is inserted.

Particularly preferably, the electromagnetic rotary drive with the rotor 10 and the stator 100 is designed as a temple motor, wherein the stator 100 has a plurality of coil cores 125, each of which comprises a longitudinal leg 126 extending from a first end in axial direction A to a second end, as well as a transverse leg 127 which is arranged at the second end of the longitudinal leg 126 and in the radial plane E. The transverse leg 127 extends in the radial direction from the longitudinal leg 126 inwards towards the rotor 10.

All first ends of the longitudinal legs 126—i.e. the lower ends according to the representation—are connected to one another by a back iron 122 for conducting the magnetic flux.

The coil cores 125 are arranged around the rotor 10 with respect to the circumferential direction, so that the rotor 10 is arranged between the transverse legs 127 of the coil cores 125. At least one concentrated winding 160 is provided at each longitudinal leg 126, which winding surrounds the respective longitudinal leg 126.

The electromagnetic fields required for the magnetic drive and the magnetic levitation of the rotor 10 are generated with the concentrated windings 160. With these concentrated windings 160, those electromagnetic fields are thus generated in the operating state with which a torque is effected on the rotor 10 in a manner known per se, and with which an arbitrarily adjustable transverse force can be exerted on the rotor 10 in the radial direction, so that the radial position of the rotor 10, i.e. its position in the radial plane E perpendicular to the axial direction A, can be actively controlled or regulated. With respect to three further degrees of freedom, namely its position in the axial direction A and tilting with respect to the radial plane E perpendicular to the desired axis of rotation (two degrees of freedom), the rotor 10 is passively magnetically levitated or stabilized by reluctance forces, i.e. it cannot be controlled.

What is claimed:

1. A pump unit for a centrifugal pump, the centrifugal pump including the pump unit and a stator extending in an axial direction from a first axial end to a second axial end, a cup-shaped recess provided at the first axial end, into which the pump unit is capable of being inserted, the pump unit comprising:
a pump housing with an inlet and with an outlet for a fluid to be conveyed; and
a rotor arranged in the pump housing with a plurality of vanes configured to convey the fluid, the rotor rotatable about the axial direction, the pump unit configured for a non-contact magnetic levitation of the rotor and for a non-contact magnetic drive of the rotor by the stator, the plurality vanes of the rotor arranged around a central inlet area of the rotor, the rotor comprising at least one relief opening to generate a recirculation flow directed from a back side of the rotor facing away from the inlet in a direction of the central inlet area of the rotor, and the rotor further having a separating element arranged in the central inlet area, the separating element configured to redirect the recirculation flow in a radial direction perpendicular to the axial direction, the plurality of vanes, the separating element and the at least one relief opening of the rotor being a one-piece unit.

2. The pump unit according to claim 1, wherein the at least one relief opening includes exactly one relief opening connecting the central inlet area of the rotor to the back side of the rotor.

3. The pump unit according to claim 1, wherein the at least one relief opening includes several relief openings arranged around a center axis of the rotor, and each relief opening of the several relief openings connecting the central inlet area of the rotor to the back side of the rotor.

4. The pump unit according to claim 1, wherein the rotor comprises a ring-shaped or disk-shaped magnetically effective core, and a sheathing completely enclosing the magnetically effective core, and the sheathing is a component of the one-piece unit, which comprises the vanes and the separating element.

5. The pump unit according to claim 1, wherein the separating element is configured and arranged such that the at least one relief opening is partially visible from the inlet.

6. The pump unit according to claim 1, wherein the separating element comprises a separating plate and attachment webs, the separating plate has a maximum outer diameter in the radial direction, which is at most as large as a diameter of the central inlet area of the rotor, and the attachment webs are configured to fix the separating plate.

7. The pump unit according to claim 6, wherein the maximum outer diameter of the separating plate is smaller than the diameter of the central inlet area of the rotor.

8. The pump unit according to claim 6, wherein the rotor comprises a ring-shaped or disk-shaped magnetically effective core, and a sheathing completely enclosing the magnetically effective core, and the sheathing is a component of the one-piece unit, which comprises the vanes and the separating element, and each attachment web of the attachment web extends from the separating plate to the sheathing, and a radial opening to recirculate flow (is disposed between adjacent attachment webs of the attachment webs.

9. The pump unit according to claim 8, wherein each attachment web of the attachment webs extends from a lower side of the separating plate in the axial direction to the sheathing.

10. The pump unit according to claim 8, wherein each attachment web is arranged at the outer edge of the separating plate and extends from the outer edge in the radial direction.

11. The pump unit according to claim 10, wherein the attachment webs are arranged equidistantly on an outer edge of the separating plate.

12. The pump unit according to claim 10 wherein each attachment web of the attachment webs extends in the radial direction to one of the plurality of vanes.

13. The pump unit according claim 10, wherein a number of attachment webs is equal to a number of vanes.

14. A centrifugal pump for conveying a fluid, comprising:
the pump unit according to claim 1;
a cylindrical cup configured to receive the rotor; and
the stator extending in the axial direction from the first axial end to the second axial end, the cup-shaped recess disposed at the first axial end, into which the cylindrical cup of the pump unit is capable of being inserted, the stator together with the rotor forming an electromagnetic rotary drive to rotate the rotor about the axial direction (A), the stator being a bearing and drive stator with which the rotor is capable of being magnetically driven without contact and capable of being magnetically levitated without contact with respect to the stator, the rotor passively magnetically stabilized with respect to the axial direction and is actively magnetically levitated in a radial plane perpendicular to the axial direction.

15. The centrifugal pump according to claim 14, wherein the electromagnetic rotary drive is a temple motor, the stator has a plurality of coil cores, each oil cord of the plurality of coil cords comprises a longitudinal leg extending from a first end in the axial direction to a second end, and a transverse leg arranged at the second end of the longitudinal leg and in the radial plane and extending from the longitudinal leg in the radial direction, the plurality of coil cores are arranged around the rotor with respect to a circumferential direction, so that the rotor is arranged between the transverse legs of the plurality of coil cores, and at least one concentrated winding is disposed on and surrounding the longitudinal leg of each of the plurality of coil.
